(19) United States
(12) Patent Application Publication
(10) Pub. No.: US 2012/0101528 A1
(43) Pub. Date: Apr. 26, 2012

(54) SPINOUS PROCESS IMPLANT AND METHOD OF FIXATION

(76) Inventors: John J. Souza, Waxhaw, NC (US); John M. Kapitan, Rock Hill, SC (US); Greg Smith, Ponte Vedra Beach, FL (US)

(21) Appl. No.: 13/191,035

(22) Filed: Jul. 26, 2011

Related U.S. Application Data
(60) Provisional application No. 61/400,221, filed on Jul. 26, 2010.

(51) Int. Cl. A61B 17/70; A61B 17/88

(52) U.S. Cl. 606/249; 606/279

(57) ABSTRACT

A spinous process fixation assembly and method for securing adjacent spinous processes. The assembly includes one or more lateral plates engageable with one or more insertion plates, respectively. The one or more lateral plates may include a material collection zone for insertion of biologic material prior to implantation. One lateral plates may be rotatable with respect to an adjacent lateral plate. One lateral plates may be movable with respect to an adjacent lateral plate between an extended and a compressed position. A single fixation device may be used to secure spinous processes across two or more levels and promote distraction at each level.

### CROSS-REFERENCE TO RELATED APPLICATION

This application claims the benefit of U.S. Provisional Patent Application Ser. No. 61/400,221, entitled SPINOUS PROCESS FIXATION SYSTEM and filed Jul. 26, 2010, the entire disclosure of which is incorporated herein by reference, to the extent that it is not conflicting with the present application.

### BACKGROUND

Common spinal surgeries include the full or partial removal of fibrous discs to relieve severe back pain. After this and other types of procedures, a surgery may secure adjacent vertebrae by either fusing the adjacent vertebrae by use of mechanical devices or fixing the vertebrae in a spaced relation if the disc space is maintained. One such type of common procedure is a lumbar inter-body fusion (LIF). A conventional device for this procedure is the use of pedicle screws and securing rods. Devices of this type require lengthy implantation time and limit spine and muscle flexibility.

More recently, implant devices have been introduced which secure to the spinous processes of two adjacent vertebrae. Most typical devices include one plate disposed on a common side of two adjacent vertebrae and a second plate on an opposing side. These types of spinous process implant devices avoid the uses of anchoring screws. However, the devices have several limitations, including limited flexibility to the surgeon during implantation.

### SUMMARY

The present application describes various exemplary methods and apparatus for a spinous process fixation system.

In an exemplary embodiment, a spinous process fixation assembly includes a first plate, a second plate, and a locking mechanism. The first plate has an inwardly facing surface and a first aperture therein. The second plate has an outwardly facing surface, an inwardly facing surface, an inwardly projecting spacer and a protrusion projecting inwardly from the spacer. The protrusion is insertable into the first aperture. The second plate is lockable by the locking mechanism in a position relative the first plate. The spacer is a cylinder-shaped cage defining at least one opening in communication with an internal material collection zone. The cage is arranged for placement of biologic material into the material collection zone prior to implantation.

In another exemplary embodiment, a spinous process fixation assembly includes a first plate, a second plate, and a locking mechanism. The first plate has a first aperture therein having a first ribbed surface. The second plate has an inwardly projecting protrusion having a second ribbed surface and insertable into the first aperture. The second plate is lockable by the locking mechanism in a position relative to the first plate. Insertion of the inwardly projecting protrusion into the first aperture provides a perceptible indication of contact between the first ribbed surface and the second ribbed surface.

In another exemplary embodiment, a spinous process fixation assembly includes a first lateral plate having a first aperture therein, a second lateral plate adjustably connected to the first lateral plate and having a second aperture therein, a first insertion plate having an inwardly projecting protrusion insertable into the first aperture, a second insertion plate having an inwardly projecting protrusion, insertable into the second aperture, a first locking mechanism and a second locking mechanism. The first insertion plate is lockable by the first locking mechanism in a position relative to the first lateral plate. The second insertion plate is lockable by the second locking mechanism in a position relative to the second lateral plate. The second lateral plate may be rotatable with respect to the first lateral plate. The second lateral plate may be movable with respect to the first lateral plate between an extended position and a compressed position.

An exemplary method of implanting a spinous fixation assembly along at least three adjacent spinous processes includes: providing an assembly including a first lateral plate having a first aperture therein, a second lateral plate adjustably connected to the first lateral plate, the second lateral plate having a second aperture therein, a first insertion plate having an inwardly projecting protrusion insertable into the first aperture, a second insertion plate having an inwardly projecting protrusion insertable into the second aperture, a first locking mechanism and a second locking mechanism, wherein the first insertion plate is lockable by the first locking mechanism in a position relative the first lateral plate and the second insertion plate is lockable by the second locking mechanism in a position relative the second lateral plate; positioning the first lateral plate along a first side of a first and second spinous processes with the first aperture oriented toward the first and second spinous processes; positioning the first insertion plate along an opposing side of a first and second spinous processes with the inwardly projecting protrusion oriented toward the first and second spinous processes; inserting the inwardly projecting protrusion of the first insertion plate into the first aperture; positioning the second lateral plate along a first side of a second and third spinous processes with the second aperture oriented toward the second and third spinous processes; positioning the second insertion plate along an opposing side of a second and third spinous processes with the inwardly projecting protrusion oriented toward the second and third spinous processes; inserting the inwardly projecting protrusion of the second insertion plate into the second aperture; locking the first insertion plate in position relative the first lateral plate; and locking the second insertion plate in a position relative the second lateral plate.

### BRIEF DESCRIPTION OF THE DRAWINGS

Features and advantages of the general inventive concepts will become apparent from the following detailed description made with reference to the accompanying drawings.

FIG. 1 is a lateral view of part of the lumbar section of a human spine;

FIG. 2 is a lateral view of part of the lumbar section of a human spine, showing a spinous process fixation assembly in an installed position;

FIG. 3 is a perspective posterior view of the lumbar section of a human spine, showing the spinous process fixation assembly of FIG. 2 in an installed position;

FIG. 4 is a posterior view of FIG. 3;

FIG. 5 is a perspective exploded view of the spinous process fixation assembly of FIG. 2;

FIG. 6 is a perspective view of a portion of the spinous process fixation assembly of FIG. 2, showing a wing;

FIG. 7 is a perspective view of the spinous process fixation assembly of FIG. 2;

FIG. 8 is a perspective view of another spinous process fixation assembly;

FIG. 9 is a perspective view of a portion of the spinous process fixation assembly of FIG. 8, showing a lateral plate;

FIG. 10 is a posterior perspective view of part of the lumbar section of a human spine, showing the spinous process fixation assembly of FIG. 8 in an installed position;

FIG. 11 is a perspective view of another spinous process fixation assembly;

FIG. 12 is a perspective view of another spinous process fixation assembly;

FIG. 13 is a lateral view of part of the lumbar section of a human spine, showing a multi-level spinous process fixation assembly in an installed position;

FIG. 14 is a perspective posterior view of FIG. 13;

FIG. 15 is a perspective exploded view of the spinous process fixation assembly of FIG. 13;

FIG. 16 is a perspective front view of the spinous process fixation assembly of FIG. 13;

FIG. 17 is a perspective rear view of the spinous process fixation assembly of FIG. 13;

FIG. 18 is an perspective view of a portion of the spinous process fixation assembly of FIG. 13, showing a ball joint component of a multi-level lateral plate;

FIG. 19 is a perspective posterior view of the lumbar section of a human spine, showing the multi-layer spinous process fixation assembly of FIG. 13 in a partially installed position;

FIGS. 20-23 are perspective posterior views of the lumbar section of a human spine, showing another multi-layer spinous process fixation assembly in a partially installed position and having wings of various structure;

FIG. 24a is a perspective posterior view of the lumbar section of a human spine, showing the multi-layer spinous process fixation assembly of FIG. 13 in a partially installed position;

FIG. 24b is a lateral view of the FIG. 24b;

FIG. 24c is a perspective posterior view of the lumbar section of a human spine, showing the multi-layer spinous process fixation assembly of FIG. 13 in a partially installed position and the multi-level lateral plate in an extended position;

FIG. 24d is a perspective posterior view of the lumbar section of a human spine, showing the multi-layer spinous process fixation assembly of FIG. 13 in a partially installed position and the multi-level lateral plate in a retracted position;

FIG. 25a is perspective view of a portion of a spinous process fixation assembly, showing a wing;

FIG. 25b is a plan view of a portion of the wing of FIG. 25a;

FIG. 26a is a perspective posterior view of the lumbar section of a human spine, showing another multi-layer spinous process fixation assembly in a partially installed position;

FIG. 26b is a posterior view of the FIG. 26a;

FIG. 27a is a perspective posterior view of the lumbar section of a human spine, showing another multi-layer spinous process fixation assembly in a partially installed position;

FIG. 27b is a perspective exploded view of multi-layer spinous process fixation assembly of FIG. 27a;

FIG. 27c is a posterior view of the lumbar section of a human spine, showing another spinous process fixation assembly in an installed position;

FIG. 28a is a perspective view of a lateral plate and an installation tool;

FIG. 28b is a perspective view of a lateral plate and an installation tool in an engaged position;

FIG. 29a is a perspective view of a lateral plate and an installation tool in an engaged position, and a wing and an installation tool in a non-engaged position;

FIG. 29b is another perspective view of a FIG. 29a;

FIG. 30a is a perspective posterior view of the lumbar section of a human spine, showing a lateral plate and an installation tool in an engaged position, and a wing and an installation tool in an engaged position;

FIG. 30b is a posterior view of FIG. 30a, showing the lateral plate in an installed orientation;

FIG. 30c is a posterior view of FIG. 30a, showing the lateral plate and the wing in an installed orientation;

FIG. 30d is a lateral view of FIG. 30c, showing a set screw and an installation tool in an engaged position; and

FIG. 30e is a lateral view of FIG. 30c, showing the set screw and installation tool in an installation position.

### DETAILED DESCRIPTION

This Detailed Description merely describes exemplary embodiments in accordance with the general inventive concepts and is not intended to limit the scope of the invention in any way. Indeed, the invention as described by the claims is broader than and unlimited by the exemplary embodiments set forth herein, and the terms used herein have their full ordinary meaning.

The general inventive concepts will now be described with occasional reference to the exemplary embodiments of the invention. This general inventive concept may, however, be embodied in different forms and should not be construed as limited to the embodiments set forth herein. Rather, these embodiments are provided so that this disclosure will be thorough and complete, and will fully convey the scope of the general inventive concepts to those skilled in the art.

Unless otherwise defined, all technical and scientific terms used herein have the same meaning as commonly understood by one of ordinary skill in the art encompassing the general inventive concepts. The terminology set forth in this detailed description is for describing particular embodiments only and is not intended to be limiting of the general inventive concepts. As used in this detailed description and the appended claims, the singular forms “a,” “an,” and “the” are intended to include the plural forms as well, unless the context clearly indicates otherwise.

Unless otherwise indicated, all numbers expressing quantities of ingredients, properties such as molecular weight, reaction conditions, and so forth as used in the specification and claims are to be understood as being modified in all instances by the term “about.” Accordingly, unless otherwise indicated, the numerical properties set forth in the specification and claims are approximations that may vary depending on the suitable properties sought to be obtained in embodiments of the present invention. Notwithstanding that the numerical ranges and parameters setting forth the broad scope of the general inventive concepts are approximations, the numerical values set forth in the specific examples are reported as precisely as possible. Any numerical values, however, inherently contain certain errors necessarily resulting from error found in their respective measurements.

The invention is directed to a spinous process implant to aid in fusion and stabilization in one or more spine levels during a posterior access surgery, while providing distraction at each level. The implant can be used as a stand-alone device, or as a supplement to another device. The implant is adjustable to multi-level angulation, i.e., lordosis and kyphosis, and to rotational abnormalities of the patient anatomy. The implant can be pre-loaded with optional biologic material to strengthen fixation by promoting bone fusion. Features of the invention allow the device to adjust to a variety of combinations of angulations, lengths, translations, and rotations of the patient anatomy during single or multiple level surgery.

The mature human spine is made up of approximately 24 vertebrae, each composed of several parts that act as a whole to surround and protect the spinal cord and nerves, provide structure to the body and enable fluid movement in many planes. The anatomy of the spine may be described in three major sections: the cervical, the thoracic, and the lumbar spine. Each section is made up of individual bones called vertebrae, e.g., the five lumbar vertebrae. An individual vertebrae is made up of several parts including the body of the vertebrae, which is the primary area of weight bearing and provides a resting place for the fibrous discs which separate each of the vertebrae, and the spinous process, which point backward and downward and are the part of the spine felt from the back of a human.

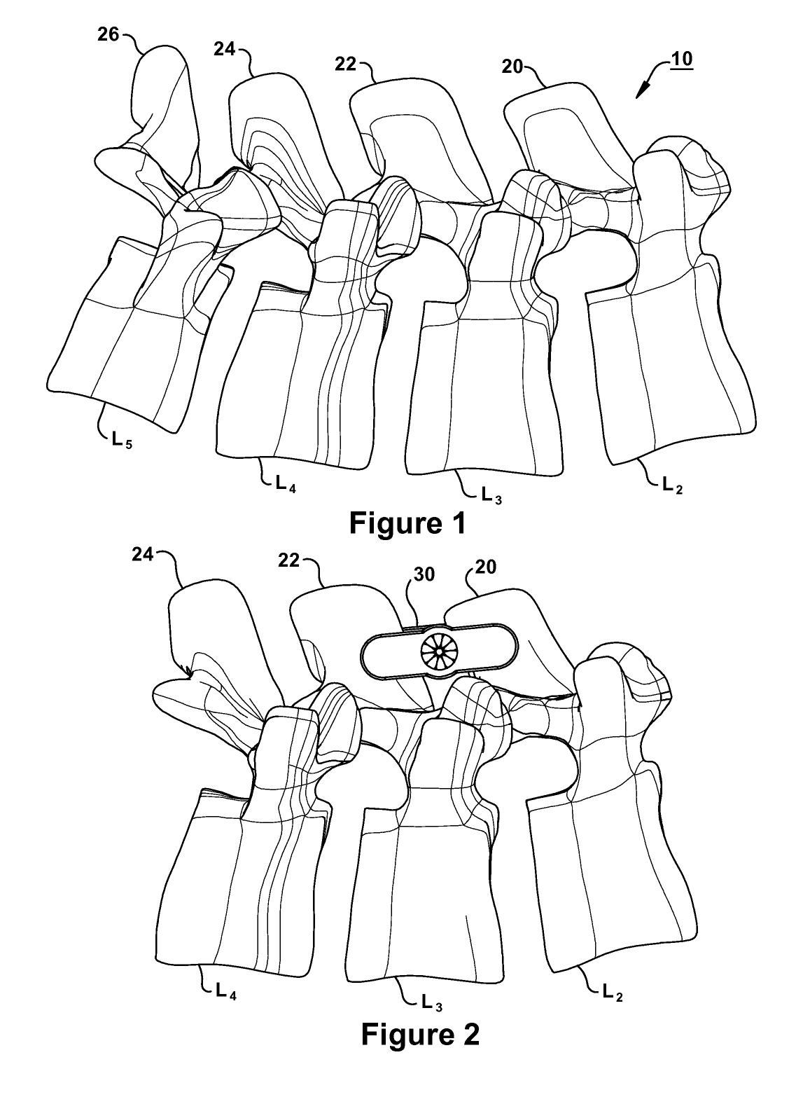

For purposes of discussion only, the invention will be described in an implantation environment of the lumbar section of the spine. It should be understood the invention has application beyond the environment described and illustrated herein, including but not limited to, the L5-S1 spine environment. Referring now to the drawings, FIG. 1 is a lateral view of part of an exemplary lumbar section 10 of a human spine. Specifically, FIG. 1 shows the second, third, fourth and fifth lumbar vertebrae L2, L3, L4, and L5, in series orientation from head to pelvis. For clarity only, the vertebrae are illustrated without discs between each pair of adjacent vertebrae. The spinous process 20, 22, 24, 26 of each lumbar vertebrae L2, L3, L4, and L5, respectively, is illustrated pointing backward and downward.

Components of the spinous fixation assembly are light weight and of sufficient strength. Exemplary materials include metals, such as for example, titanium alloys and cobalt alloys, and plastics, such as for example, PEEK. The materials may be biocompatible to allow the use of biologic material in implantation, such as for example, bone grafts, bone marrow, and other tissue samples.

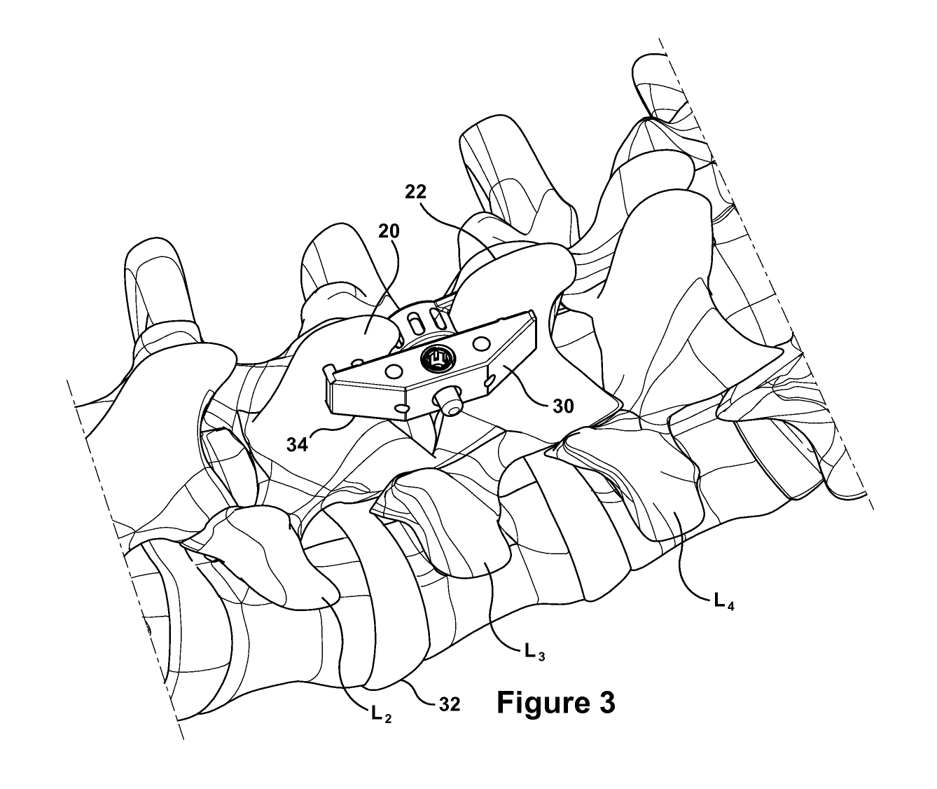

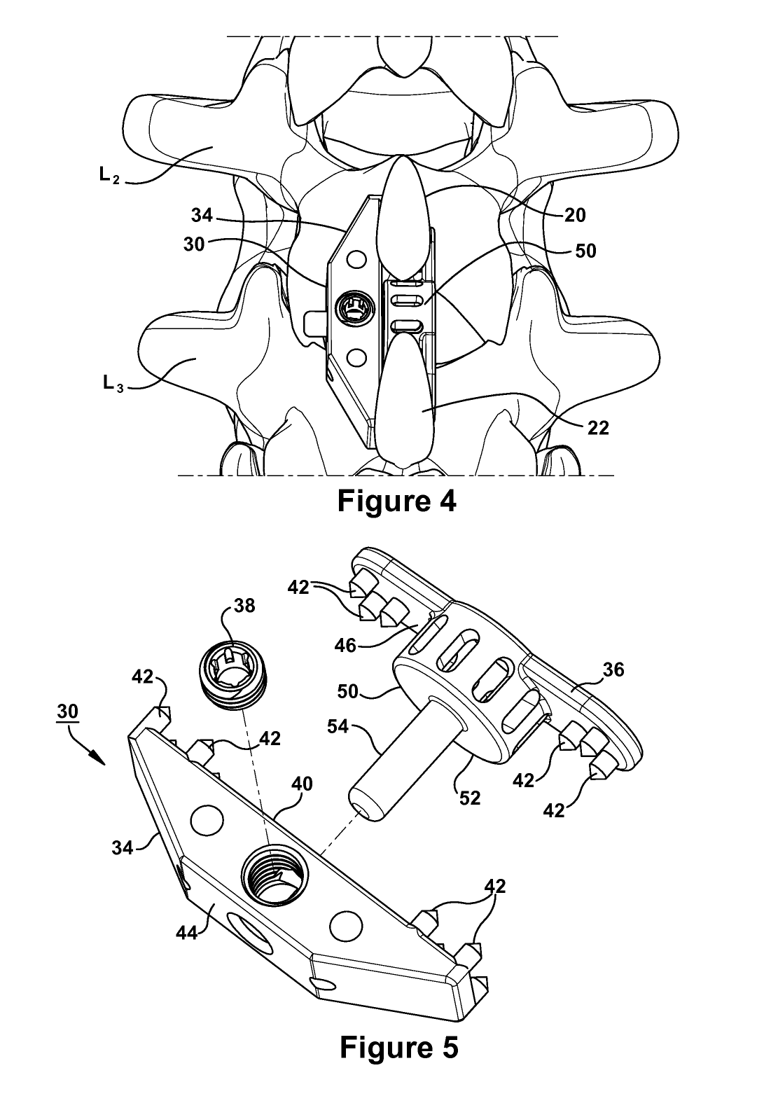

An embodiment of the invention is applicable for a single level application. Referring to FIG. 2, a lateral view of part of the lumbar section of FIG. 1 is shown, with an exemplary spinous process fixation assembly 30 in an installed position. A perspective posterior view and a posterior view of the spinous process fixation assembly 30 in an installed position are shown in FIGS. 3 and 4, respectively. For exemplary purposes only, FIGS. 3 and 4 illustrate the fibrous disc 32 between the second lumbar level L2 and third lumbar level L3. The resultant distraction of L2 and L3 is best shown in FIG. 4.

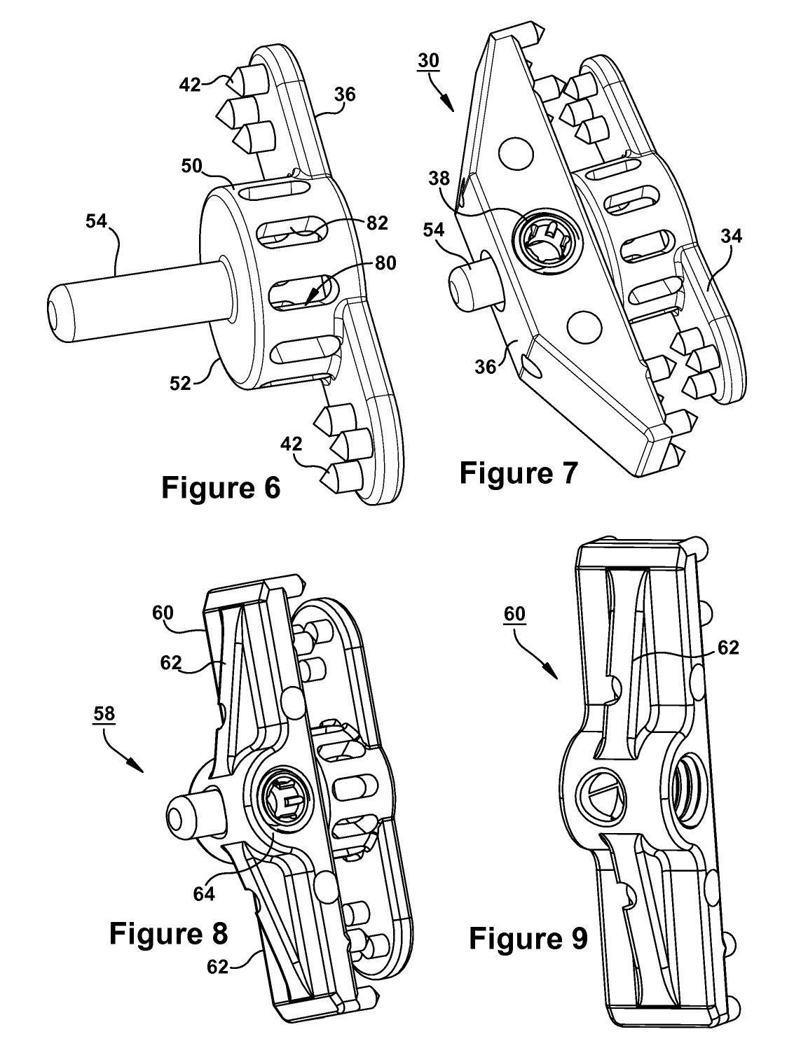

The spinous process fixation assembly provides flexibility to the surgeon during implantation. Specifically, the assembly 30 is structured for a surgeon to manipulate components into desired position relative to the spinal anatomy of the patient. A perspective exploded view of the spinous process fixation assembly 30 is shown in FIG. 5. As illustrated, the assembly 30 includes a first plate 34, a second plate 36, and a locking mechanism 38. An additional perspective view of the second plate 36 is shown in FIG. 6.

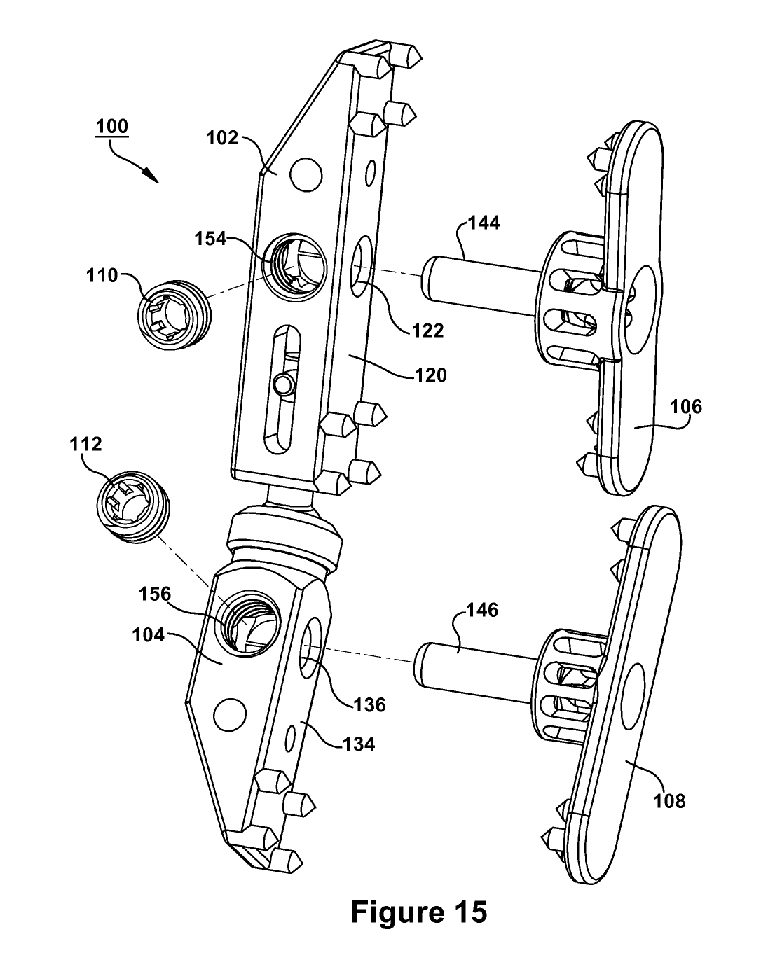

As shown in FIGS. 3-4, the first plate 34 is sized for parallel placement along one side of two adjacent vertebrae. The first plate 34, or a lateral plate, has an having an inwardly facing surface 40 which is orientated toward the vertebrae. The first plate may be sized to one of various lengths to accommodate a patient's anatomy, such as vertebrae of various size and interspinous process distraction. One or more teeth 42 protrude from the surface of the first plate 34. The teeth may be of any functional pattern or number. During implanting, the teeth engage tissue and bone to aid fixation. The inwardly facing surface further includes a first aperture (not shown). The aperture may be of various size or shape, such as for example, a round hole, a slot, or a slot with a ridged inner surface. However, the aperture is of a size and shape and characteristic to allow insertion of a protrusion of the second plate 36. Exemplary apertures are shown in FIGS. 15, 23 and 27b. When the aperture is a slot, the slot may have a width greater than a width of the inwardly projecting protrusion of the second plate. In this arrangement, the inwardly projecting protrusion is insertable at a plurality of locations within the slot, giving the surgeon increased flexibility during implantation.

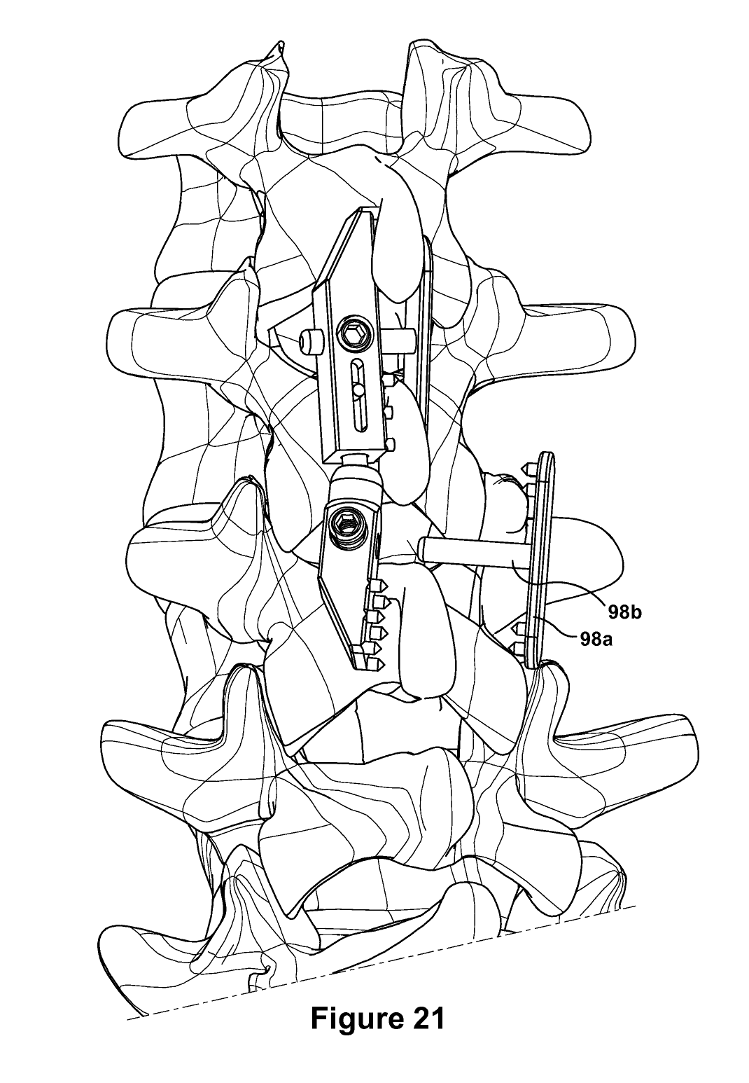

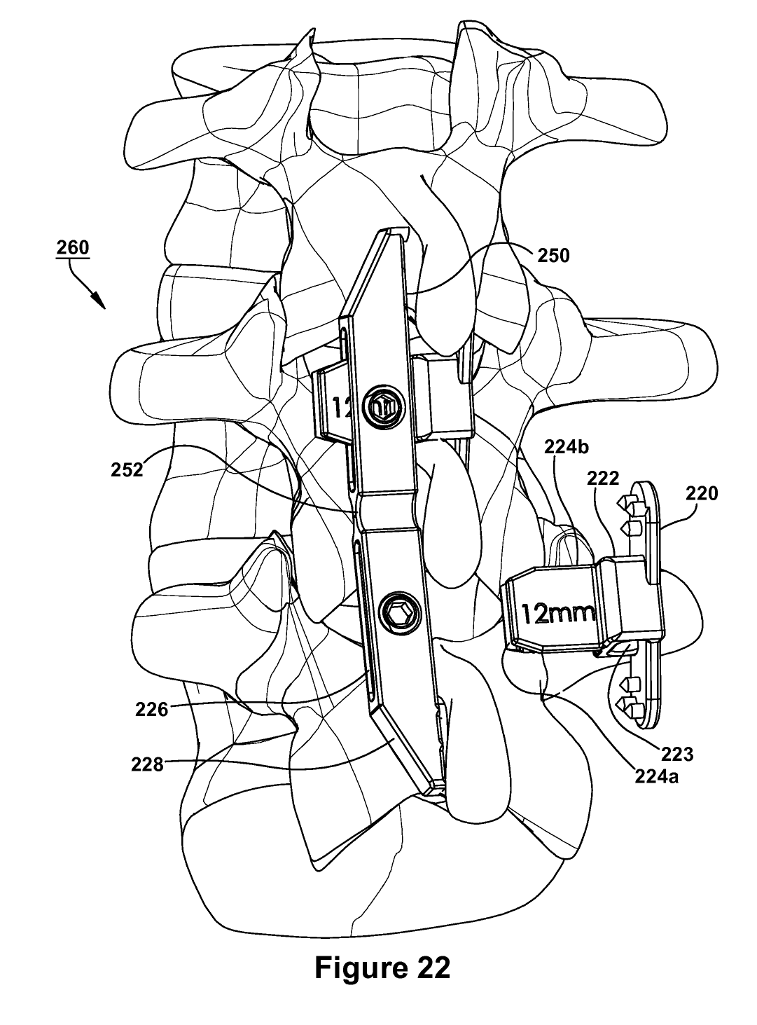

As discussed, a second plate 36 is included in the implant assembly 30. The second plate, or a wing, has an inwardly facing surface 46 relative the vertebrae. The second plate may be sized to one of various lengths to accommodate patient's anatomy, such as vertebrae of various size and interspinous process distraction. Similar to the first plate, one or more teeth 42 protrude from the surface of the second plate 36. A open barrel-like spacer 50 also protrudes inwardly toward the vertebrae. As shown, the spacer is a metal alloy and non-compressible, thus during implantation, the inner surface 52 of the spacer 50 impedes second plate movement to minimize the distance between the first and second plates 34, 36. The spacer maybe cylinder-shaped as shown in FIG. 6, a block, or any suitable shape. To be discussed in greater detail, an exemplary second plate 220 having a block-shaped spacer 222 is shown in FIG. 22. The spacer 222 defines a covered void 223 suitable for placement of material, to be discussed in further detail. With the implant installed, the spacer provides distraction between two adjacent vertebrae. In comparison, a second plate 98a is illustrated having a rod 98b, but absent a spacer, is illustrated in FIG. 21. In this embodiment, distraction is achieved by maintaining the adjacent vertebrae in a spaced relation by fixation of the teeth.

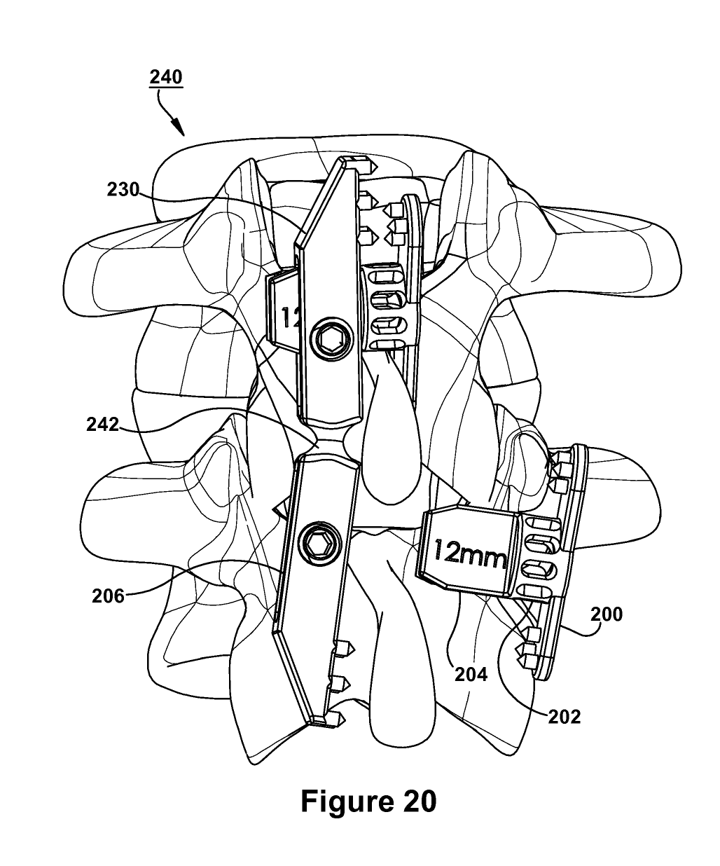

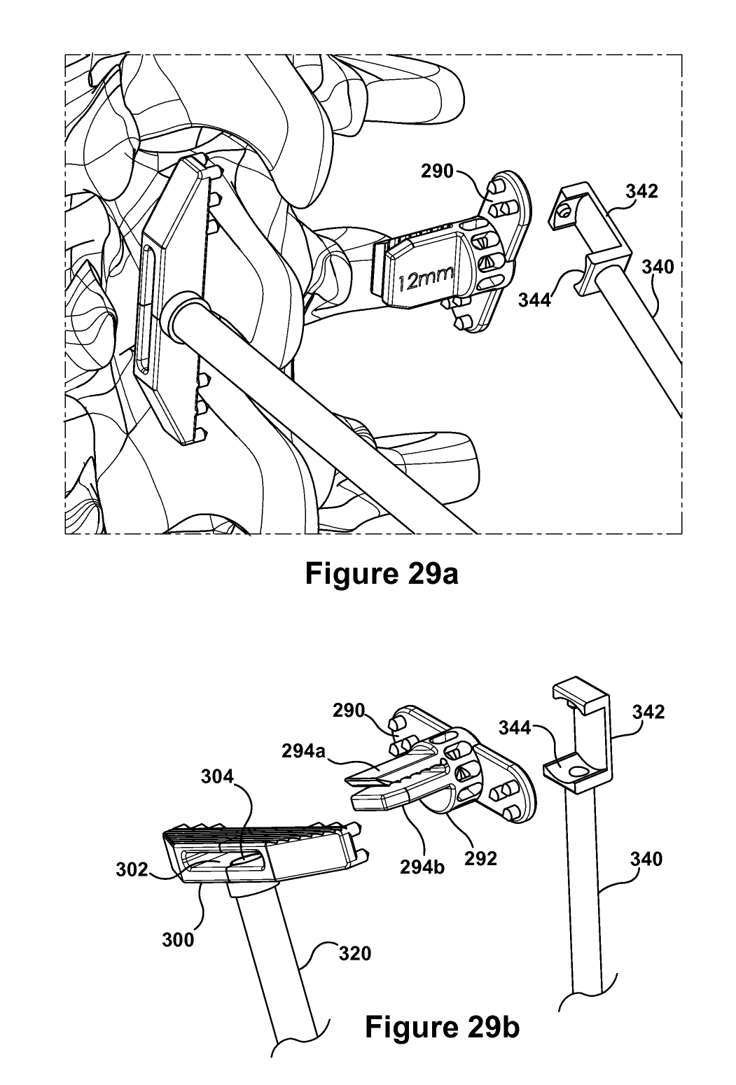

The plates 34, 36 are joined by the insertion of an inwardly projecting protrusion 54 into the first aperture of the first plate. As shown in FIGS. 5 and 6, the protrusion is a rod 54. The rod is sized for friction free insertion into the first aperture of the first plate. A surgeon may rotate the second plate 36 relative the first plate 34 after insertion and prior to compressing the plates 34, 36 into tissue and bone. The rod protrusion may be other sizes and shapes, such as for example, a tab sized for insertion into a slot as shown in FIG. 20, a tab with ridges on its contact surface as shown in FIG. 29b.

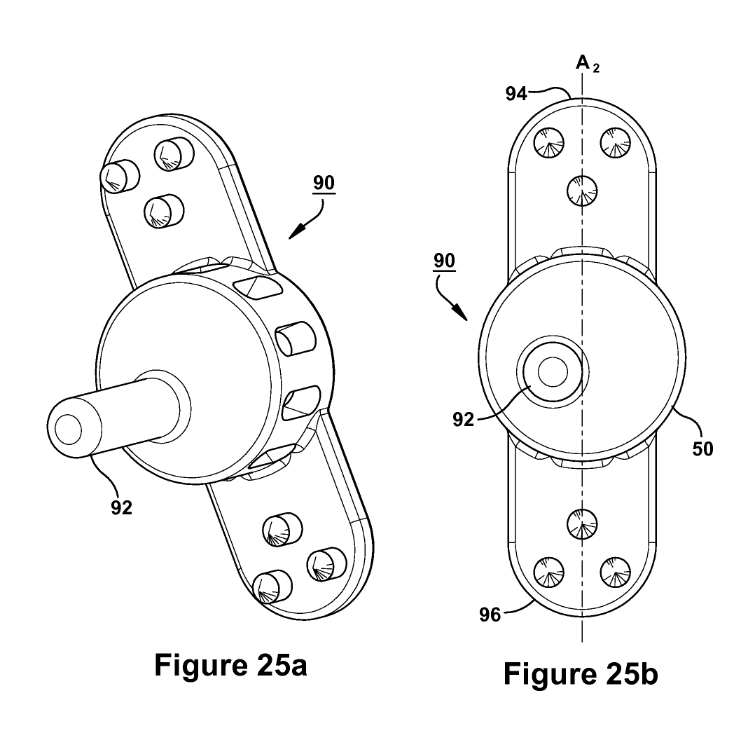

The position of the rod 54 of the inwardly facing surface may also vary in practice of the invention. FIGS. 25a and 25b are perspective and plan views, respectively, of another embodiment of a second plate. The second plate 90 includes a rod 92 protruding from the spacer off-center from a longitudinal axis A₁ of the second plate. The illustrated off-center position is for example only. In this arrangement, the second plate 90 is insertable into a companion first plate in at least two parallel positions relative the first plate. For example, the first plate is inserted in the orientation shown in FIG. 25b, with the first end 94 at the top and the second end 96 at the bottom. If the second plate 90 was inserted into a companion first plate in the reverse orientation, with the second end 96 at the top and the first end at the bottom, the second plate 90 would still be parallel to the first plate but would be shifted to the right to a different relative position. This off-center placement of the rod 92 provides the surgeon with increased flexibility during implantation.

Upon insertion of the protrusion into the first aperture, a surgeon may use the locking mechanism to lock the second plate in a position relative the first plate. FIG. 7 is a perspective view of the resulting spinous process fixation assembly 30. As shown, a self locking set screw 38 is inserted into a threaded hole 44 on the first plate and tightened to prohibit movement of the rod 54.

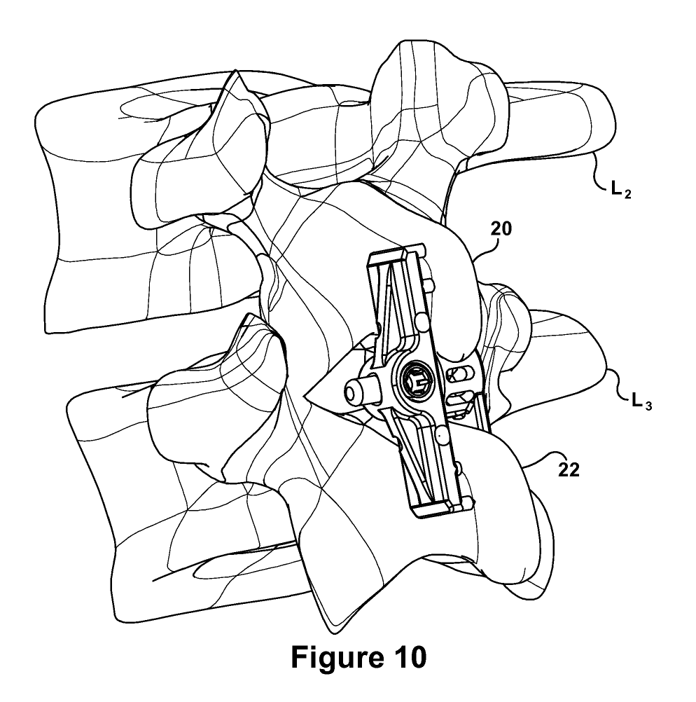

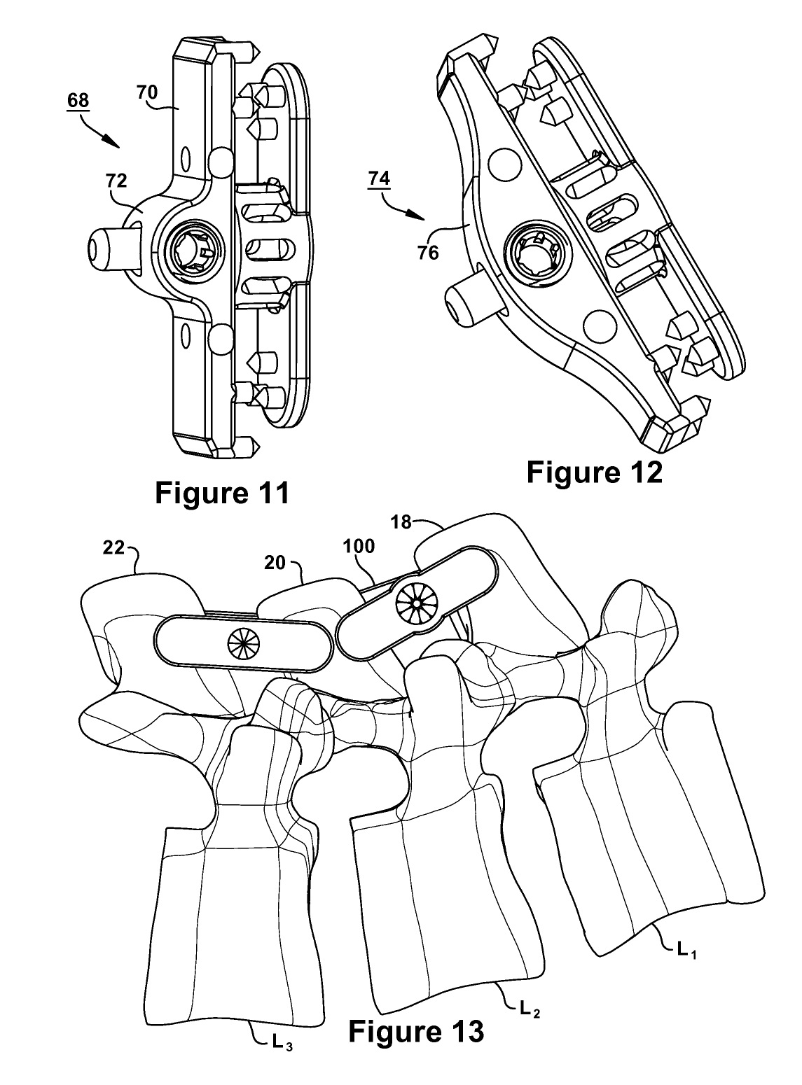

FIGS. 8-10 illustrated another embodiment of the first plate. As shown in FIG. 8, an implant assembly 58 includes a first plate 60. The first plate 60 has an outwardly facing surface defining two ribs 62 raising to a center arched section 64. FIG. 10 shows the assembly 58 in an implanted position. Additional embodiments of first plates are shown in implant assemblies 68,74 in FIGS. 11 and 12, respectively. Implant assembly 68 includes a first plate 70 having a center arched section 72 between two flat outwardly facing surfaces. The first plate assembly 74 of the first plate 76 has an arcuate outwardly facing surface with a raised center between opposing ends.

The invention allows the fixation assembly to strengthen and bond to the adjacent vertebrae after implantation by providing for use of biologic material. Specifically, a surgeon may deposit biologic material, such as bone marrow, in the assembly prior to implantation. After the implant is secured in the desired position and the surgery is completed, the biological material will grow and bond with the surrounding spinal bone and tissue, resulting is a stronger fusion of the subject vertebrae.

Referring again to FIG. 6, the illustrated spacer 50 defines at least one material collection zone 80. The spacer 50 is arranged as a cylinder-shaped cage with a set of radial slots 82. In the practice of the invention, one skilled in the art will appreciate the zone or zones may take on any suitable shape or style, such as for example, a concave or ridged surface in the second plate. The zone is suitable for surgeon placement of biologic material prior to implantation, for example, by use of a needle through one or more slots 82. However, the surgeon could also place the material into the zone just after the first plate had been joined with the second plate. Also, the surgeon could place the material into the zone through an opening on an outwardly facing surface of the first plate 36. For example, FIG. 17 illustrates an insertion plate 106 having an outwardly facing surface 140a defining an opening 140b. After implantation, the slots 82 allow for outward movement or growth of biologic material from the collection zone 80.

Another embodiment of the invention promotes ease of implantation for the surgeon through perceptible indications of mating between the second plate and the first plate. Specifically, the insertion of the inwardly projecting protrusion of the second plate into the first aperture of the first plate provides a perceptible indication of contact. The perceptible indication may be from tactile, such as between a first ribbed surface of the first plate and a second ribbed surface of the second plate. The perceptible indication of contact may be in another form of resistance or may be audible. Upon the perceptible indication, a surgeon is aware the implant has been provisionally locked with the first plate engaged to the second plate. In this condition, the surgeon may proceed to lock the implant against the subject vertebrae. Alternatively, certain embodiments allow further position adjustment of the plates prior to final locking.

An embodiment of the invention is applicable for multiple level applications with a single implant. The single implant offers the surgeon multiple component flexibility in the cephalad/caudal direction, angular flexibility between adjacent levels, increased fixation strength and reliability, and decreased implantation time as compared to the use of multiple single level devices.

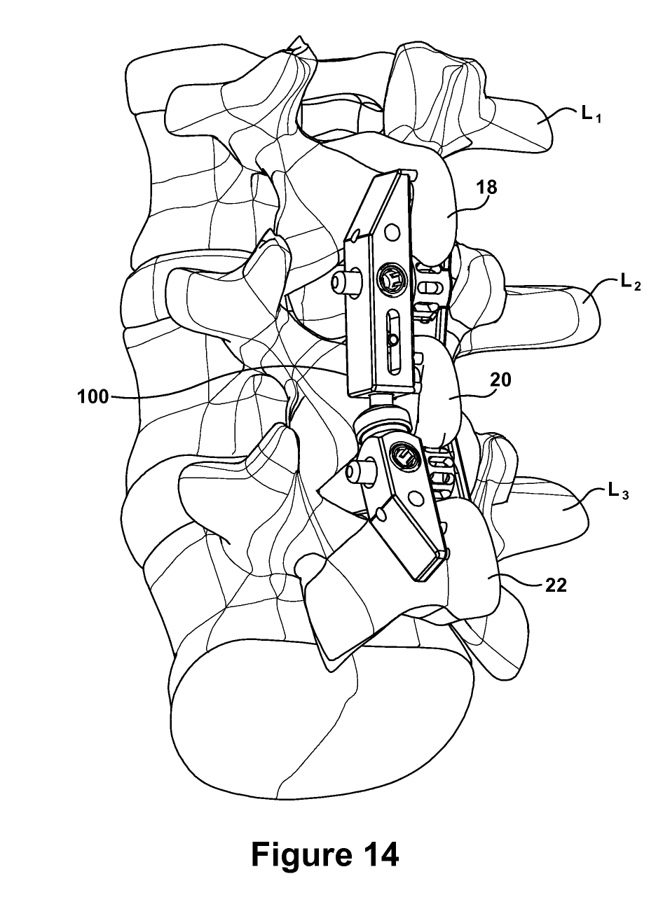

An embodiment of a multi-level spinous process fixation assembly 100 is illustrated in FIGS. 13-17. A lateral and perspective posterior view of the implant 100 in an implanted position is shown in FIGS. 13 and 14, respectively. As shown, lumbar L1, L2 and L3 are illustrated in a fixed and distracted position. FIGS. 15-17 will be referenced for discussion of the components of the implant 100.

The implant 100 includes generally a first lateral plate 102, a second lateral plate 104, a first insertion plate 106, a second insertion plate 108, a first locking mechanism 110 and a second locking mechanism 112. To be discussed in greater detail, the implant 100 offers the surgeon flexibility during implantation, including adjustability between the position of the first lacteal plate 102 and the position of the second lateral plate 104. Specifically, the implant 100 offers flexibility of the overall implant length and relative rotational positioning of the first and second lateral plates 102, 104.

The first lateral plate 102 shares some structural features as the single level lateral plate 34 previously discussed. The first lateral plate 102 is sized for parallel placement along one side of two adjacent vertebrae, e.g., L1 and L2 in FIG. 14. The first lateral plate 102 has an inwardly facing surface 120 which is orientated toward the vertebrae. A first aperture 122 accommodates insertion of a protrusion from the first insertion plate 106.

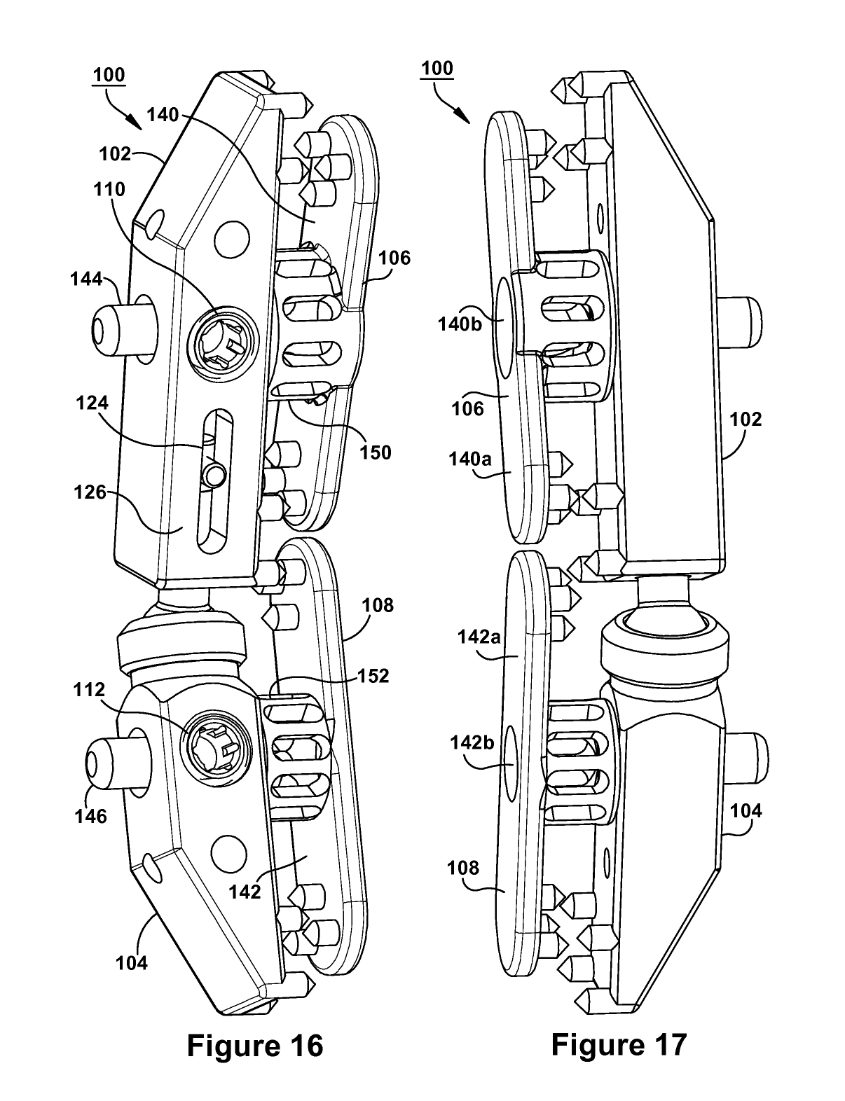

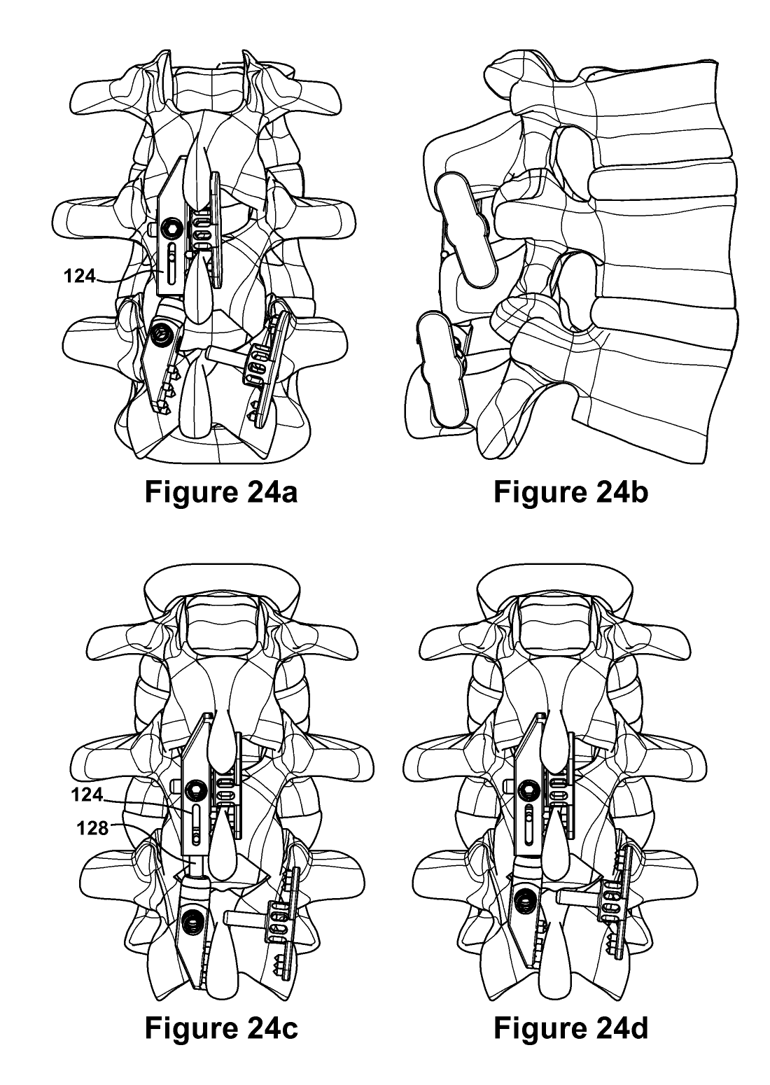

As best seen in FIG. 16, the first lateral plate 102 includes a slot and piston joint to allow the surgeon to adjust the first and second lateral plates 102, 104 between and extended position (see FIG. 24c) and an compressed position (see FIG. 24d). The slot 124 is within a posterior facing surface 126 to accommodate a piston 128, illustrated in FIG. 18. The piston 128 includes a tab 130 which rides within the outer portion of the slot. The opposing end of the piston includes a ball joint 132 which connects the second lateral plate 104 to the first lateral plate 102, and allows rotational motion of the second lateral plate 104 relative the first lateral plate 102.

The second lateral plate 104 also shares some structural features as the single level lateral plate 34 previously discussed. The second lateral plate 104 is sized for parallel placement along one side of two adjacent vertebrae, e.g., L2 and L3 in FIG. 14. The first lateral plate 102 has an inwardly facing surface 134 which is orientated toward the vertebrae. A second aperture 136 accommodates insertion of a protrusion from the second insertion plate 108.

As discussed best seen in FIG. 16, the first lateral plate 102 includes a slot and piston joint to allow the surgeon to adjust the first and second lateral plates 102, 104 between and extended position (see FIG. 24c) and an compressed position (see FIG. 24d). The slot 124 is within a posterior facing surface 126 to accommodate a piston 128, illustrated in FIG. 18. The piston 128 includes a tab 130 which rides within the outer portion of the slot. The opposing end of the piston includes a ball joint 132 which allows rotational motion of the second lateral plate 104 relative the first lateral plate 102. Other rotational joints may be used in the practice of the invention. A ball joint and other types of rotational joints allow a surgeon to accommodate for variations in patient anatomy, including lateral curvature.

Another embodiment of multi-level implant allows for rotational movement between the first lateral plate and the second lateral plate. FIG. 20 shows an exemplary implant 240 in a fixed position. The implant includes a first lateral plate 230 and a second lateral plate 206. The first and second lateral plates 230, 206 are a single uniform piece, e.g., a machined titanium alloy. The first and second lateral plates 230, 206 include a bendable portion 242 of reduced cross-section. A surgeon may rotate the second lateral plate about this bendable portion 242 when the implant is in the position shown in FIG. 20. This bendable portion is advantageous when a surgeon wishes to rotate the second lateral plate in one particular plane, but not in any other plane.

Another embodiment of multi-level implant allows for rotational movement between the first lateral plate and the second lateral plate in a common plane. FIG. 22 shows an exemplary implant 260 in a fixed position. The implant includes a first lateral plate 250 and a second lateral plate 228. The first and second lateral plates 250, 228 are a single uniform piece, e.g., a machined titanium alloy. The first and second lateral plates 250, 228 include a bendable portion 252 of reduced cross-section. A surgeon may swing the second lateral plate about this bendable portion 252 when the implant is in the position shown in FIG. 22. In this arrangement, the second lateral plate 228 swings in a common plane with respect to the first lateral plate 250.

Returning to FIGS. 12-17, the exemplary first and second insertion plates 106, 108 as illustrated are essentially the same in structure as the previously discussed wing 36 of the single level device. Each insertion plate 106, 108 has an inwardly facing surface 140, 142, and outwardly facing surface 140a, 142a, and an inwardly projecting protrusion 144, 146. The outwardly facing surface 140a, 142a define opening 140b, 142b leading to material collection zones with the spacer. As illustrated, the protrusion 144, 146 is insertable into and rotatable within the companion aperture 122, 136. As previously discussed, one or both rods may be disposed off-center from the longitudinal axis of the insertion plate.

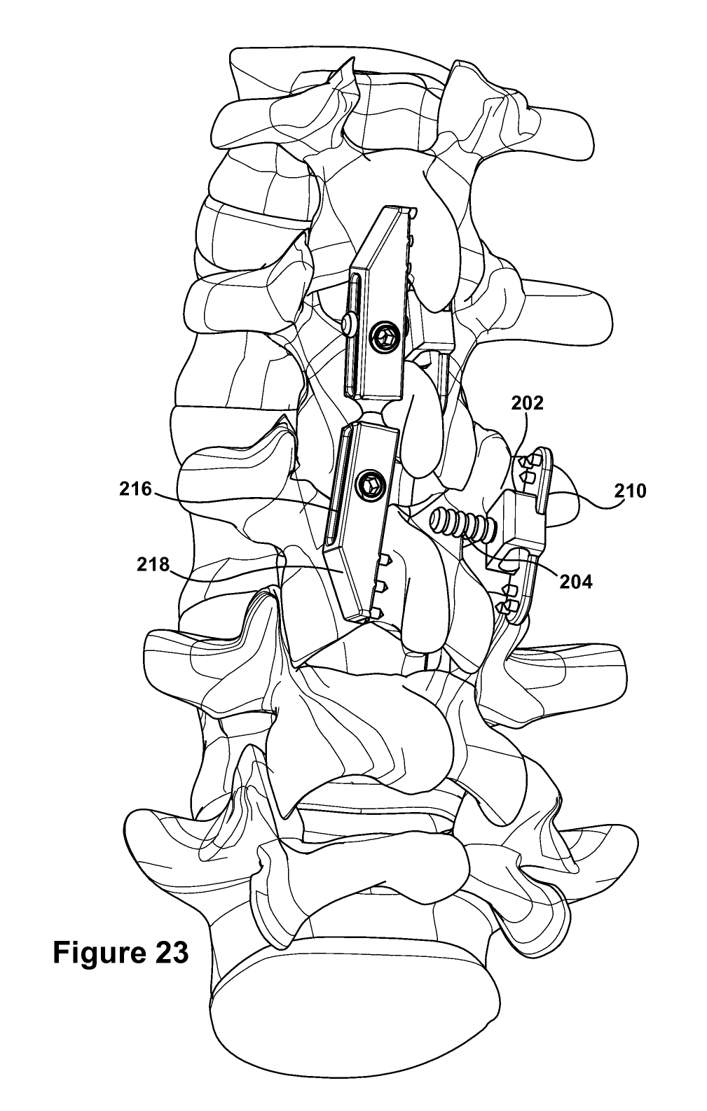

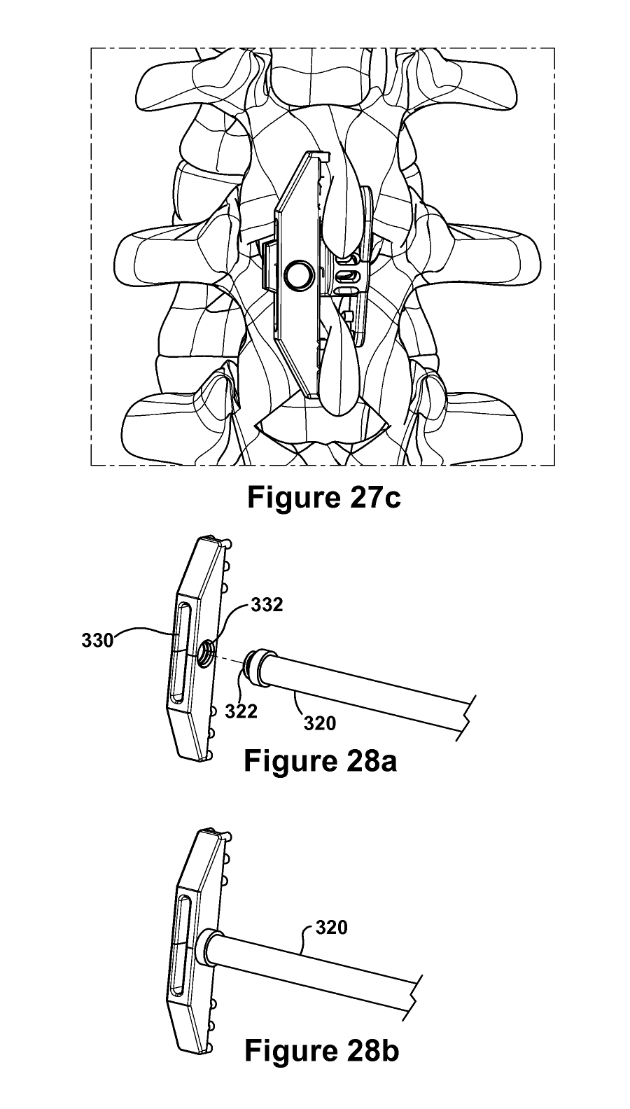

One embodiment of multi-level implant includes a protrusion with a tab design. FIG. 20 shows a second insertion plate 200 which includes a cage style spacer 202 and a single tab 204. The tab is sized for insertion into a cooperatively sized slot (not shown) in a second lateral plate 206. Another embodiment shown in FIG. 23 includes a protrusion with a rod design. As illustrated, a second insertion plate 210 includes a void or tunnel block style spacer 202 and a ridged rod 204. The rod is sized for insertion into a cooperatively sized slot 216 in a second lateral plate 218. The slot 216 extends a sufficient length of the second lateral plate 218 to allow a surgeon to insert the rod in various locations and at various angles. Another embodiment shown in FIG. 22 includes a protrusion with a twin tab design. As illustrated, a second insertion plate 220 includes a void or tunnel block style spacer 222 and a twin tab 224a, 224b. The tabs are sized for insertion into a cooperatively sized slot 226 in a second lateral plate 228. One of the twin tabs penetrates the slot and the other is positioned on the posterior or anterior surface of the lateral plate, as best seen in the exemplary insertion shown in FIG. 27b. The slot 226 extends a sufficient length of the second lateral plate 228 to allow a surgeon to insert the rod in various locations. Exemplary tabs and slots with ridges or laminations are shown in FIGS. 27b and 29b.

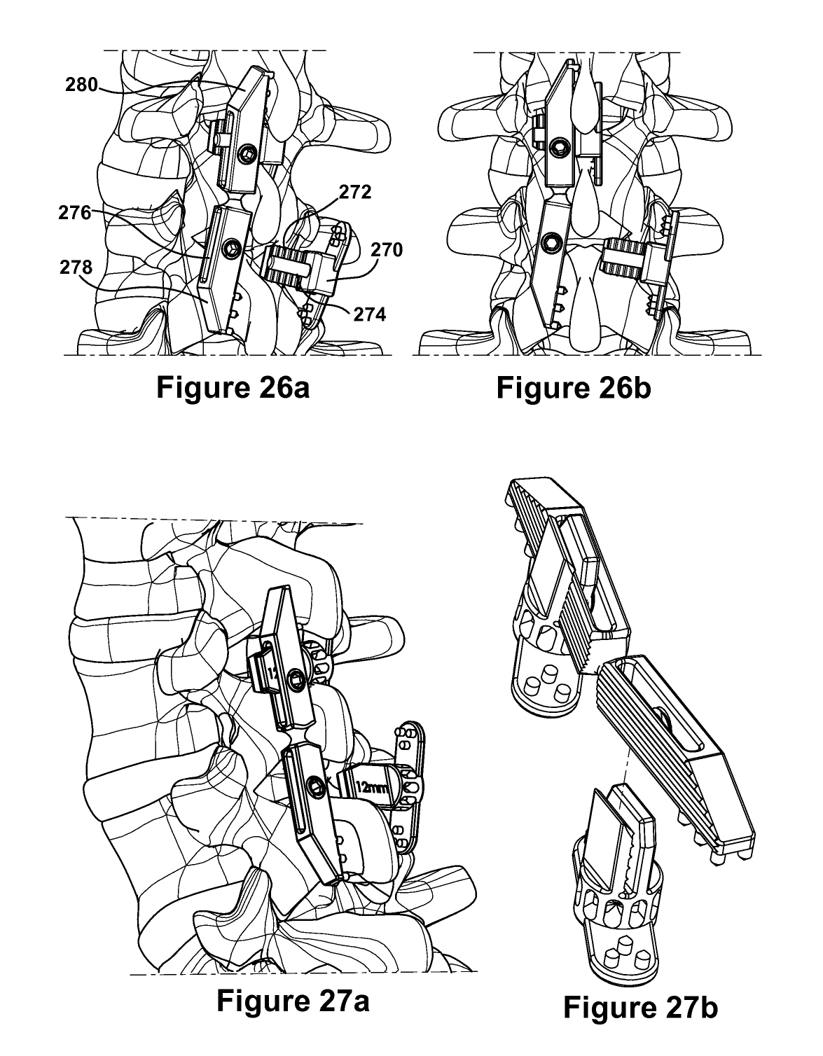

Yet another embodiment shown in FIG. 26a includes a double protrusion design. A second insertion plate 270 includes a ridged tab 272 and a rod 274. The rod is sized for insertion at a surgeon-determined location within a slot 276 in a second lateral plate 278. The ridged tab is positioned on the posterior or anterior side of the second lateral plate. The inserted position is best seen in the locked condition of the first lateral plate 280 in FIG. 26a.

Another embodiment of a double protrusion arrangement is shown in FIG. 29a. An insertion plate 260 includes a flat tab 294a and a ridged tab 294b, each protruding from a cage spacer 292. The flat tab 294a is sized for insertion at a surgeon-determined location within a slot 302 in a lateral plate 300. The ridged tab 294b is positioned on a ridged side 304 of the lateral plate 300.

Referring again to FIG. 16, the insertion plates 106, 108 also include a spacer 150, 152 essentially the same as the previously discussed single level spacer 50. Each spacer 150, 152 is a cylinder-shaped cage defining a material collection zone. The zone is suitable for pre-loading of biologic material by a surgeon. The open cage design allows growth of biologic material from the zone after implantation.

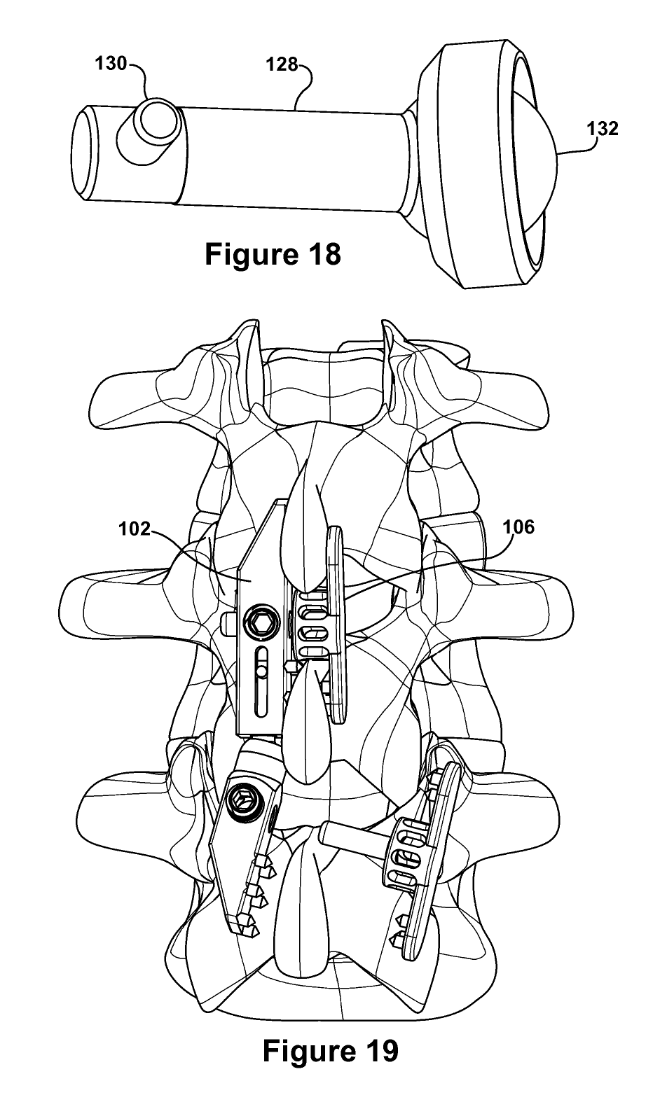

Again similar to the single level device, a first locking mechanism and second locking mechanism are used to secure the implant 100 in a locked position. Upon insertion of the first protrusion 144 into the first aperture 122 as shown in FIG. 19, a surgeon may use the first locking mechanism to lock the first insertion plate 106 in a position relative the first lateral plate 102. A self locking set screw 110 is inserted into a threaded hole 154 on the first lateral plate and tightened to prohibit movement of the rod 144. After axial adjustment by a surgeon using the slot and piston joint, and rotational adjustment using the ball joint, the surgeon may join the second insertion plate 108 with the second lateral plate 104, and insert a self locking set screw 112 into a threaded hole 156 on the second lateral plate 104 and tightened to prohibit movement of the rod 146.

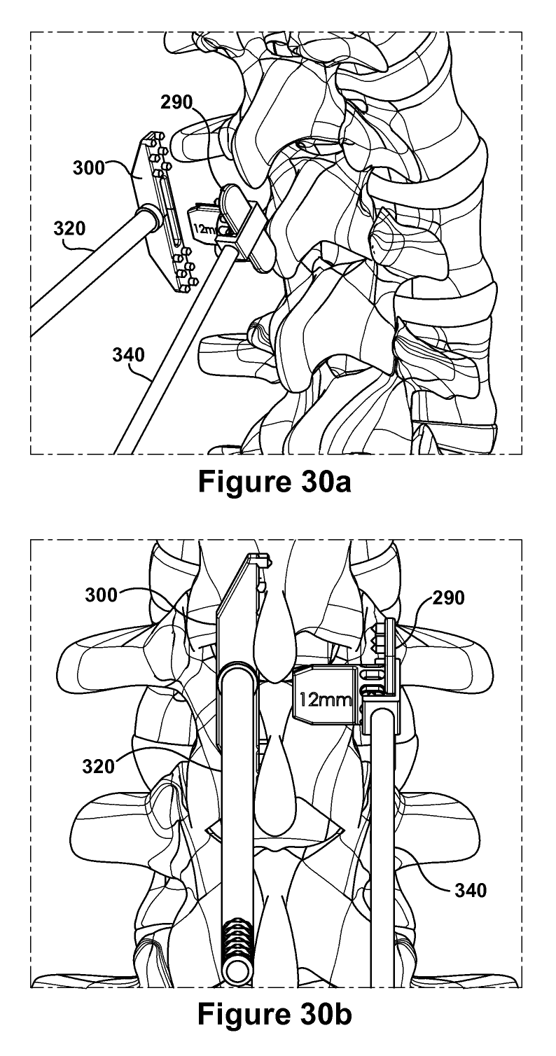

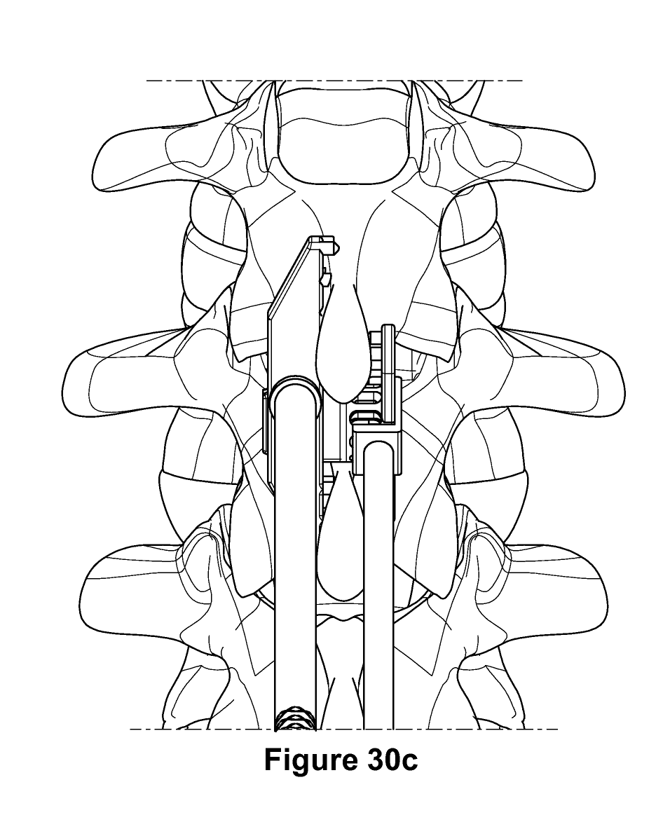

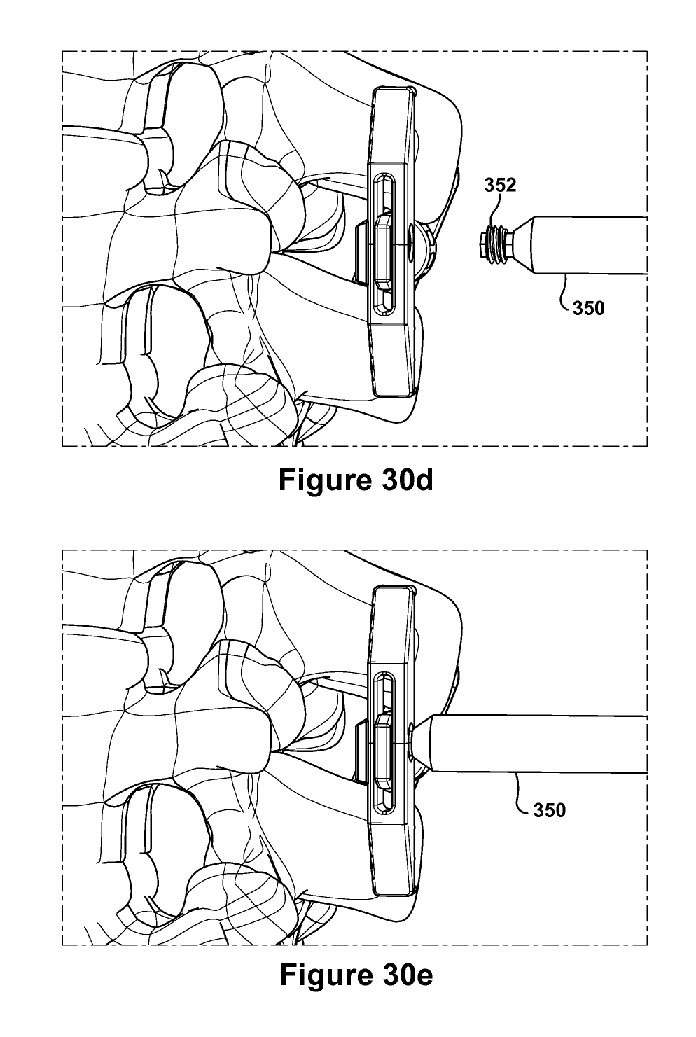

The inventive spinous process implant includes features to ease installation by a surgeon using simple instrumentation. The use of tools to aid in implantation is illustrated in FIGS. 28a-30e. Specifically, FIGS. 28a and 28b illustrate the use of an elongated rod 320 to control and manipulate a lateral plate 330. A surgeon may thread a male end 322 of the rod 320 into a hole 332 on the posterior side of the lateral plate. Likewise, FIG. 29a illustrates an insertion plate holding tool 340. The tool 340 includes a bracket 342 with an arcuate inner surface 344 sized to enclosed a portion of the outer surface of a cage spacer. Once the spacer is secured, a surgeon may elect this point in the surgical procedure to pre-load the optional biologic material. FIGS. 30a and 30b shown the tools 320, 340 holding the plates 320, 290 prior to implantation. By further use of the tools 320, 340, or a compressor tool, the surgeon may manipulate the plates into the desired orientation and engage the plates as shown in FIG. 30c. This step will result in engagement of the implant teeth with the bone. When the lateral plate is engaged and provisionally locked with the insertion plate, tools 320, 340 are removed and a locking tool 350 is used to deliver and secure the self locking screw 352 as shown in FIGS. 30d and 30e to lock the lateral plate 300 in place using the same hole 332. An exemplary load of 80 lbs may be applied.

A method of the invention includes an implantation procedure along at least three adjacent spinous processes. The method includes providing a single multi-level implant as described herein. An exemplary implant in shown in FIGS. 15 and 16. After securing the plate with instrumentation, a surgeon positions the first lateral plate along a first side of a first and second spinous processes with the first aperture oriented toward the first and second spinous processes. A first insertion plate is then positioned along an opposing side of a first and second spinous processes with the inwardly projecting protrusion oriented toward the first and second spinous processes. The inwardly projecting protrusion of the first insertion plate is inserted into first aperture. This step provisionally locks the plates together and may provide the surgeon with a perceptible indication, e.g., audible, that mating has occurred between the plates. A surgeon then positions the second lateral plate along a first side of a second and third spinous processes with the second aperture oriented toward the second and third spinous processes. A second insertion plate is then positioned along an opposing side of a second and third spinous processes with the inwardly projecting protrusion oriented toward the second and third spinous processes. The inwardly projecting protrusion of the second insertion plate is inserted into the second aperture. This step provisionally locks the plates together and may also provide the surgeon with a perceptible indication of mating. To complete fixation, the first insertion plate is locked in position relative the first lateral plate, and the second insertion plate is locked in a position relative the second lateral plate.

The method may include steps offering the surgeon with improved flexibility in implantation. A surgeon may rotate the second lateral plate relative the first lateral plate prior to insertion of the inwardly projecting protrusion of the second insertion plate into second aperture. For example, the surgeon may rotate the second lateral plate by use of a ball joint as shown in FIG. 19.

Another process step that may be utilized by the surgeon offers flexibility regarding the position of the second lateral plate with respect to the fist lateral plate along the spine. A surgeon may move the second lateral plate with respect to the first lateral plate between an extended position and a compressed position prior to insertion of the inwardly projecting protrusion of the second insertion plate into second aperture. For example, the surgeon may move the second lateral plate relative the first lateral plate by use of a piston and slot joint as shown in FIG. 16. The joint in the extended position is shown in FIG. 24c and the compressed position is shown in FIG. 24d. The joint is adjustable between these two positions.

The method may include the use of additional material with the implant to strengthen the fusing of the subject vertebrae after implantation is completed. Preferably, the method includes pre-loading at least one material collection zone in one of the first insertion plate and the second insertion plate with biologic material. Preferably, the collection zone is defines by a cage-style spacer as shown in FIG. 16. The method may include strengthening the connection between two adjacent spinous processes by allowing growth of the biologic material beyond the at least one material collection zone after implantation.

While various inventive aspects, concepts and features of the general inventive concepts are described and illustrated herein in the context of various exemplary embodiments, these various aspects, concepts and features may be used in many alternative embodiments, either individually or in various combinations and sub-combinations thereof. Unless expressly excluded herein all such combinations and sub-combinations are intended to be within the scope of the general inventive concepts. Still further, while various alternative embodiments as to the various aspects, concepts and features of the inventions (such as alternative materials, structures, configurations, methods, circuits, devices and components, software, hardware, control logic, alternatives as to form, fit and function, and so on) may be described herein, such descriptions are not intended to be a complete or exhaustive list of available alternative embodiments, whether presently known or later developed. Those skilled in the art may readily adopt one or more of the inventive aspects, concepts or features into additional embodiments and uses within the scope of the general inventive concepts even if such embodiments are not expressly disclosed herein. Additionally, even though some features, concepts or aspects of the inventions may be described herein as being a preferred arrangement or method, such description is not intended to suggest that such feature is required or necessary unless expressly so stated. Still further, exemplary or representative values and ranges may be included to assist in understanding the present disclosure; however, such values and ranges are not to be construed in a limiting sense and are intended to be critical values or ranges only if so expressly stated. Moreover, while various aspects, features and concepts may be expressly identified herein as being inventive or forming part of an invention, such identification is not intended to be exclusive, but rather there may be inventive aspects, concepts and features that are fully described herein without being expressly identified as such or as part of a specific invention. Descriptions of exemplary methods or processes are not limited to inclusion of all steps as being required in all cases, nor is the order that the steps are presented to be construed as required or necessary unless expressly so stated.

What is claimed is:

1. A spinous process fixation assembly comprising:
a first plate having an inwardly facing surface and a first aperture therein;
a second plate having an outwardly facing surface, an inwardly facing surface, an inwardly projecting spacer and a protrusion projecting inwardly from the spacer, the protrusion insertable into the first aperture; and
a locking mechanism, wherein the second plate is lockable by the locking mechanism in a position relative the first plate;
wherein the spacer is a cylinder-shaped cage defining at least one opening in communication with an internal material collection zone, the cage arranged for placement of biologic material into the material collection zone prior to implantation.

2. The spinous process fixation assembly of claim 1, wherein the cage includes radial passages for outward movement of biologic material from the material collection zone to outside the cage after implantation.

3. The spinous process fixation assembly of claim 1, wherein the outwardly facing surface of the second plate includes an opening leading to the material collection zone.

4. A spinous process fixation assembly comprising:
a first plate having an inwardly facing surface and a first aperture therein having a first ribbed surface;
a second plate having an inwardly facing surface and an inwardly projecting protrusion, the protrusion having a second ribbed surface and insertable into the first aperture; and
a locking mechanism, wherein the second plate is lockable by the locking mechanism in a position relative the first plate;
wherein insertion of the inwardly projecting protrusion into the first aperture provides a perceptible indication of contact between the first ribbed surface and the second ribbed surface.

5. The spinous process fixation assembly of claim 4, wherein the first aperture is a slot having a width greater than a width of the inwardly projecting protrusion.

6. The spinous process fixation assembly of claim 5, wherein the inwardly projecting protrusion is insertable at a plurality of locations within the slot.

7. The spinous process fixation assembly of claim 4, wherein the perceptible indication is audible.

8. The spinous process fixation assembly of claim 4, wherein the perceptible indication is tactile.

9. A spinous process fixation assembly for stabilizing multiple levels of spinous processes, the assembly comprising:
a first lateral plate having an inwardly facing surface and a first aperture therein;
a second lateral plate rotatably connected to the first lateral plate, the second lateral plate having an inwardly facing surface and a second aperture therein;
a first insertion plate having an inwardly facing surface and an inwardly projecting protrusion, the protrusion insertable into the first aperture;
a second insertion plate having an inwardly facing surface and an inwardly projecting protrusion, the protrusion insertable into the second aperture;
a first locking mechanism, wherein the first insertion plate is lockable by the first locking mechanism in a position relative the first lateral plate; and
a second locking mechanism, wherein the second insertion plate is lockable by the second locking mechanism in a position relative the second lateral plate.

10. The spinous process fixation assembly of claim 9, wherein the second lateral plate is connected to the first lateral plate by a ball joint member.

11. The spinous process fixation assembly of claim 9, wherein the first lateral plate and the second lateral plate are a single uniform piece.

12. The spinous process fixation assembly of claim 11, wherein the second lateral plate is connected to the first lateral plate by a bendable portion of reduced cross-section relative the first lateral plate and the second lateral plate.

13. The spinous process fixation assembly of claim 9, wherein the second lateral plate rotates about the first lateral plate in a common plane.

14. The spinous process fixation assembly of claim 9, wherein the second lateral plate is movable with respect to the first lateral plate between an extended position and a compressed position.

15. The spinous process fixation assembly of claim 9, wherein at least one of the first insertion plate and the second insertion plate comprise a spacer defining at least one material collection zone.

16. The spinous process fixation assembly of claim 15, wherein the spacer is a cage arranged for placement of biologic material in the at least one material collection zone prior to implantation.

17. The spinous process fixation assembly of claim 16, wherein the spacer defines radial passages for outward movement of biologic material from the at least one material collection zone after implantation.

18. The spinous process fixation assembly of claim 9, wherein the protrusion of the first insertion plate is rotatable within the first aperture.

19. The spinous process fixation assembly of claim 9, wherein the protrusion of the second insertion plate is rotatable within the second aperture.

20. The spinous process fixation assembly of claim 9, wherein at least one of the first aperture and the second aperture is a slot.

21. The spinous process fixation assembly of claim 20, wherein at least one of the first insertion plate protrusion and the second insertion plate protrusion comprise a tab having a ridged surface.

22. The spinous process fixation assembly of claim 9, wherein at least one of the first insertion plate and the second insertion plate comprise a second protrusion.

23. The spinous process fixation assembly of claim 20, wherein at least one of the first insertion plate protrusion and the second insertion plate protrusion is insertable at a plurality of locations within the slot.

24. A spinous process fixation assembly for stabilizing multiple levels of spinous processes, the assembly comprising:
a first lateral plate having an inwardly facing surface and a first aperture therein;
a second lateral plate adjustably connected to the first lateral plate, the second lateral plate having an inwardly facing surface and a second aperture therein;
a first insertion plate having an inwardly facing surface and an inwardly projecting protrusion, the protrusion insertable into the first aperture;
a second insertion plate having an inwardly facing surface and an inwardly projecting protrusion, the protrusion insertable into the second aperture;
a first locking mechanism, wherein the first insertion plate is lockable by the first locking mechanism in a position relative the first lateral plate; and
a second locking mechanism, wherein the second insertion plate is lockable by the second locking mechanism in a position relative the second lateral plate.

25. The spinous process fixation assembly of claim 24, wherein the second lateral plate is movable with respect to the first lateral plate between an extended position and a compressed position.

26. The spinous process fixation assembly of claim 24, wherein the second lateral plate is secured to the first lateral plate by a piston and slot connection and slidable with respect to the first lateral plate.

27. A method of implanting a spinous fixation assembly along at least three adjacent spinous processes, the method comprising:
providing an assembly comprising a first lateral plate having a first aperture therein, a second lateral plate adjustably connected to the first lateral plate, the second lateral plate having a second aperture therein, a first insertion plate having an inwardly projecting protrusion insertable into the first aperture, a second insertion plate having an inwardly projecting protrusion insertable into the second aperture, a first locking mechanism and a second locking mechanism, wherein the first insertion plate is lockable by the first locking mechanism in a position relative the first lateral plate and the second insertion plate is lockable by the second locking mechanism in a position relative the second lateral plate;
positioning the first lateral plate along a first side of a first and second spinous processes with the first aperture oriented toward the first and second spinous processes;
positioning the first insertion plate along an opposing side of a first and second spinous processes with the inwardly projecting protrusion oriented toward the first and second spinous processes;
inserting the inwardly projecting protrusion of the first insertion plate into the first aperture;
positioning the second lateral plate along a first side of a second and third spinous processes with the second aperture oriented toward the second and third spinous processes;
positioning the second insertion plate along an opposing side of a second and third spinous processes with the inwardly projecting protrusion oriented toward the second and third spinous processes;
inserting the inwardly projecting protrusion of the second insertion plate into the second aperture;
locking the first insertion plate in position relative the first lateral plate;
locking the second insertion plate in a position relative the second lateral plate.

28. The method of claim 27, further comprising rotating the second lateral plate relative the first lateral plate prior to insertion of the inwardly projecting protrusion of the second insertion plate into second aperture.

29. The method of claim 27, further comprising moving the second lateral plate with respect to the first lateral plate between an extended position and a compressed position prior to insertion of the inwardly projecting protrusion of the second insertion plate into second aperture.

30. The method of claim 27, further comprising:
wherein at least one of the first insertion plate and the second insertion plate comprise a spacer defining at least one material collection zone;
providing biologic material within the at least one material collection zone prior to implantation.

31. The method of claim 30, further comprising:
strengthening the connection between two adjacent spinous processes by allowing growth of the biologic material beyond the at least one material collection zone after implantation.
